(19) United States
(12) Patent Application Publication
(10) Pub. No.: US 2012/0189549 A1
(43) Pub. Date: Jul. 26, 2012

(54) DEVICE FOR AUTOMATING BEHAVIORAL EXPERIMENTS ON ANIMALS

(76) Inventor: Adam Claridge-Chang, Brooklyn, NY (US)

(21) Appl. No.: 13/349,797

(22) Filed: Jan. 13, 2012

Related U.S. Application Data
(60) Provisional application No. 61/432,972, filed on Jan. 14, 2011.

(51) Int. Cl. A61K 49/00; A01K 29/00; A61B 5/00; C07K 2/00

(52) U.S. Cl. 424/9.2; 530/300; 119/712; 600/300

(57) ABSTRACT

The present invention enables testing the effect of one or more test agent(s) on one or more animal(s) of a group of animals, preferably insects, to identify agents that affect behavioral properties of the animals. The invention is comprised of the steps of providing animals suitable for testing, bringing those animals into contact with the test agent(s), moving the animals from a growth container to isolate them, prepare and separate the animals for singulation, relocated the animals to a behavior arena, subject the animals to behavioral tracking to assess their behavioral state, and removing the animals from the behavioral tracking to facilitate iterative analysis of further groups. The invention enables a method for preparing a therapeutic compound for the treatment of an animal disease.

This application claims priority of U.S. Provisional Application No. 61/432,972.

Several of the references cited in the disclosure are listed below. In addition, the following references are relevant to the present invention as is obvious from their content, which are hereby incorporated by reference herein in their entirety: U.S. Pat. Nos. 7,848,888; 5,848,571; 4,634,328; 4,692,965; 5,195,921; 6,758,323; 7,776,584; 4,106,438; 5,587,062; 6,688,255; 7,776,584; 4,388,798; 4,822,022; 3,965,608; 6,264,419; and 7,642,066; European patent numbers 1421994 B1; and 1421994 B1; PCT application WO 1992/012233 A1; Uber et. al., “Application of Robotics and Image Processing to Automated Colony Picking and Arraying,” Biotechniques, vol. 11, No. 5, 1991, pp. 642-646, XP008026697; Jones et al., “Integration of Image Analysis and Robotics Into a Fully Automated Colony Picking and Plate Handling System,” Nucleic Acids Research, vol. 20, No. 17, 1992, pp. 4599-4606, XP002190262. Furthermore, several other references are included in the disclosure that are not included in the above references.

### 1. BACKGROUND OF THE INVENTION

1.1 Field of the Invention

The present invention relates in general to the field of machines for sorting live animals, and in particular to a device for isolating, sorting and delivering insects into containers.

1.2 Description of the Related Art

Psychiatric and neurological diseases affect millions of people worldwide, and there is great interest in identifying biologically active agents that can alleviate them. One approach to try to understand and find treatments employs animal models, particularly rodents with dysfunctional behavior that parallels similar behavior in humans. However, processing rodents for testing biologically active agents is very expensive, particularly in behavioral measurements which may require specialize equipment and numerous measurements to detect a statistically significant behavioral abnormality. Insects, such as, for example, Drosophila melanogaster, have long been used to find genes and other biologically active agents that affect behavior and the brain functions that produce behavior. Using Drosophila gives significant cost savings per behavioral measurement per animal. Insects are cheaper to propagate and much smaller than rodents. The shorter propagation time of insects like Drosophila also mean that genetic studies can be completed more rapidly than rodent genetic studies. Gene homology between insects and mammals including humans mean that agents that have a behavioral effect in insects may reasonably also have an effect on mammalian behavior. Thus insect behavioral screening provides a way to identify agents that affect psychiatric or neurological illness in human.

Some assays analyse behavior in large groups of insects, for example gross locomotor behavior. However, for some behaviors, using individual flies or small groups are better approaches, due to possible complex interactions between animals that may confound the behavior, for example, courting interactions or aggression between individuals. Certain assays require individuals or small, defined groups.

However, the common approach to sorting insects, such as, for example, flies, into small groups or individuals is to manually singulate and deliver insects to the behavioral chamber and is a time-consuming process. Insects must be anesthetized, spread out on a flat surface and then transferred to the behavioral container, in the case of flies, one by one using fine-tipped forceps. The operation to be repeated to singulate flies involves:

1) Find a fly and orient the forceps so as to pinch the wings.

### 2) Pick up the fly.

3) Transfer to a container and release the fly.
4) Repeat from step 1

This task is difficult and there is an upper limit of around 2000 flies per day per person.

Furthermore, human operation leads to variable anesthesia times, potential injury to the flies with the sharp forceps and other differences that vary between experimenters and even iterations and thus introduce and extra source of procedural inconsistency.

Analysis of insect behavior is an important technique to understand brain function in healthy and diseased animals. Many behavioral assays have a requirement for a single animal to be analyzed in an arena. Two slow steps in behavioral analysis are isolating single animals and moving them into the behavior arena, something that can take a long time when many assays are needed to obtain sufficiently statistically powered results.

One method of sorting insects into defined groups or individuals uses embryo sorting, sometimes using genetically encoded fluorescent markers. A benefit of this method is that aspects of the life history of the animal can be closely controlled. However a major disadvantage of this approach is that the behavior must be assessed in the same chamber in which the animals developed; this dramatically limits the type of assay that can be performed.

### 1.2.1. Screening for Drug Targets, Lead Compounds, and Small Molecule and Other Therapeutic Molecules.

Screening assays and techniques of various types are typically used to screen biologically active compounds for their ability to suppress or enhance a certain biological process, activity, trait, disease, disease process, etc. Cell-free and genetic assays provide, for example, identification of putative drug targets implicated in a specific disease condition, such as a specific enzymatic reaction. Cell-based assays, for example, can provide insights into mechanisms underlying disease pathogenesis, and can also provide information on possible toxicity of candidate compounds. In either case, the goal of such screening is to identify the most likely candidates or “lead compounds” for use in further drug discovery and developments efforts, and not to identify a specific drug. High throughput screening is often used to identify lead compounds or therapeutic agents from libraries consisting of large numbers of compounds. The strength of a particular screening technique lies substantially in its ability to rapidly and efficiently screen large libraries of compounds with a reasonably good rate of prediction of how an identified compound will behave in vivo, e.g. as a therapeutic agent, while remaining cost effective.

### 2. BRIEF SUMMARY OF THE INVENTION

This disclosure describes a device and method that automates behavioral analysis of animals to determine the effect of biologically active agents on their behavior. First, the device takes insects from an input container, generally the growth container, isolates them into single animals (singulation) and dispenses them to a second container, either alone or in a defined group. The machine combines the following components. Second, it possesses an animal singulator to isolate and identify single animal bodies, either while active or in an anesthetized state. Third, it has a mechanism to dispense the singulated animals. Fourth, the behavioral arena(s) is comprised within a container that is positioned to be the subject of a behavioral recording device. Fifth, in some embodiments, the behavioral arena contains or is connected to devices that deliver sensory stimuli to the animal(s). Sixth, in some preferred embodiments, the behavioral recording device and sensory stimuli are under automated control by any method known to one of ordinary skill in the art, including, but not limited to computer control. Seventh, in the embodiments that transfer anesthetized animals into the behavioral arena, the device includes an anesthesia system.

### 3. BRIEF DESCRIPTION OF THE DRAWING

The invention can be most conveniently understood by reference to the description of the preferred embodiment, together with the drawing.

FIG. 1 shows an orthogonal view of a preferred embodiment of a device for automating behavioral experiments on small insects; this preferred embodiment of the device consists of:

1. Growth containers.
2. A growth container conveyor belt.
3. A fly intake tube.
4. A fly intake cassette.
5. A cleaning cassette.
6. A clearing suction tube.
7. A suction tube.
8. Anesthesia platform.
9. Anesthesia platform conveyor.
10. Robotic rails.
11. A vertical robotic arm.
12. A behavioral plate chilling platform.
13. A Behavioral arena plate.
14. A carriage connecting vertical arm and horizontal beam.
15. A plate gripper.
16. A horizontal robot beam attached to the plate handling robot.
17. A rear horizontal rail.
18 a & b. Two behavioral inspection positions.
19 a & b. Two inspection cameras.
20. An array of independently movable fly pipets.
21. A stack of plates.
22. A stack of lids.
23. A disposal opening.
24. A front horizontal rail.
25. A horizontal robotic beam.
26. A device bed.
27. A pipetting camera.
28. A branched robotic arm.
29. A Suction connector tube.
30. An anesthesia platform wall.

### 4. DETAILED DESCRIPTION OF THE INVENTION

The present invention can be made and practiced using different embodiments of the invention. The description and depiction of a preferred embodiment of the invention in the disclosure hereinafter sets forth an example of the present invention which is not intended to limit the invention to the specific embodiment illustrated. A preferred embodiment is shown and described in detail using flies as preferred animals for use in the device and practice of the invention, however this non-limiting example is only illustrative of the invention and is not intended to limit the scope of the invention.

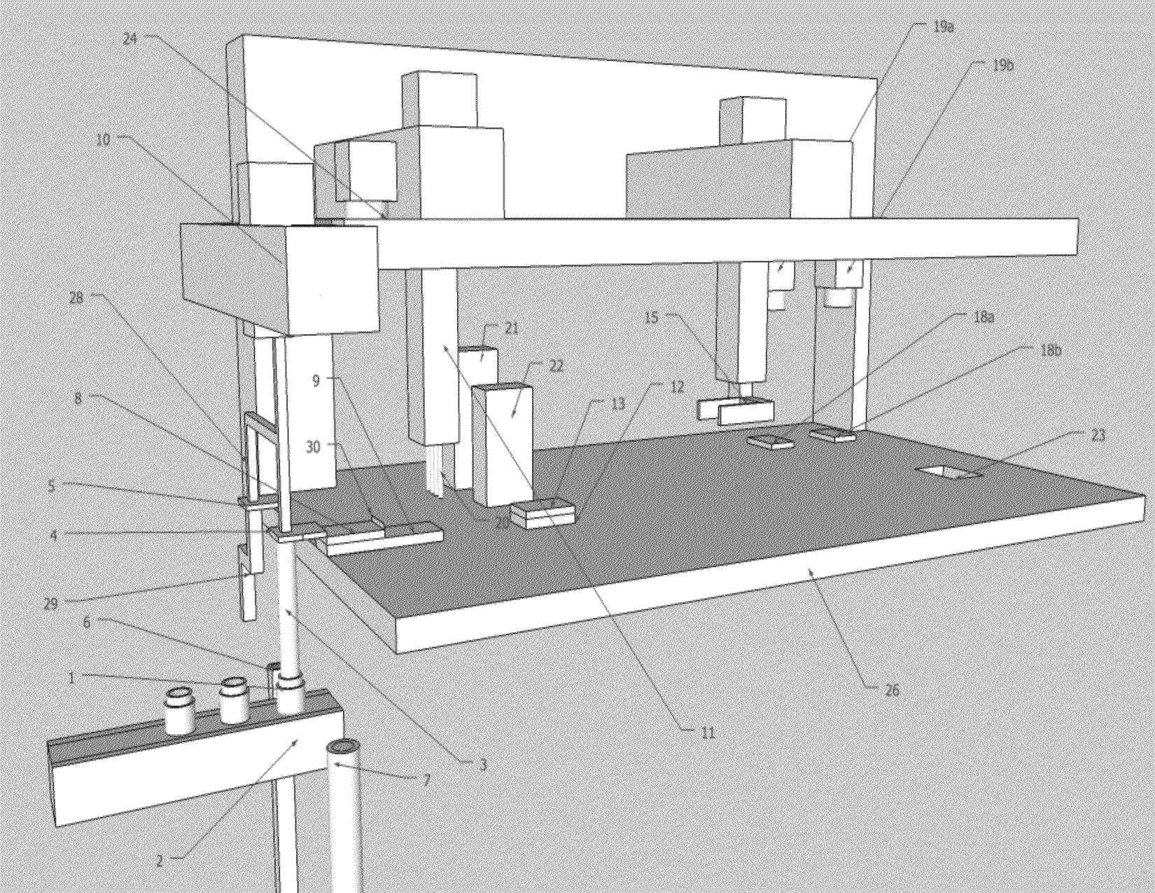

Referring to the drawing, an insect handling device for behavior is schematically illustrated in FIG. 1 and is represented in its entirety by reference numeral. FIG. 1 shows a preferred embodiment of an automated defined-individuals insect behavior system. This exemplar device works to (i) take flies from containers, and facilitate their transfer into behavioral arenas in defined numbers of individuals, including but not limited to single flies, (ii) subject singulated flies to a behavioral detection method in the behavioral arenas, including but not limited to videography, and (iii) the collect the data from the behavioral detection which can then be used to analyze the behavior.

Referring to the drawing, an exemplary embodiment is described. The invention claimed and described herein is not to be limited in scope by the specific embodiments herein disclosed since these embodiments are intended as illustrations of several aspects of the invention. Indeed, various modifications of the invention in addition to those shown and described herein will become apparent to those skilled in the art from the foregoing description. Such modifications are also intended to fall within the scope of the appended claims.

The invention as described can be applied to a variety of animals, but has particular utility when used with Drosophila melanogaster. For clarity and convenience, but not for limitation, the description generally refers to the use of flies. Also for clarity, the invention is described below first briefly as the single preferred embodiment, followed by descriptions of further exemplary embodiments and modifications of the invention. However, neither the preferred embodiment nor the modifications thereof should be construed to limit the invention.

In the preferred embodiment the invention enables testing the effects of test agent(s) on defined groups of animals, which entails acquiring a population of test animals, bringing them into contact with one or more test agents, isolating and singulating the animals, moving them into an appropriate arena for behavioral analysis, subjecting them to behavioral recording by means of video capture and computer vision analysis, removing them from the behavioral analysis area and disposing of them so as to facilitate repeated operation. In the context where multiple groups of flies are tested with multiple test agents, number of fly groups are at least 2, and often more than 2, for example at least about 10 groups, at least about 100 groups, or at least about 200 groups. Within each group, at least 3 individual flies are tested and often more, for example, at least about 10 flies, at least about 20 flies, at least about 100 flies, or at least about 500 flies. In some embodiments, the operation of the invention enables analysis of large numbers of fly groups in a day, for example at least about 10 groups, at least about 20 groups, at least about 100 groups, or at least about 200 groups.

### 4.1. The Animals to be Used

In the preferred embodiment of the present invention, a population of Drosophila melanogaster flies are used to test a series of agents for their effect on the behavior of the flies. In this simplest example, the flies are wild type flies wherein some groups have been brought into contact with test agents in, for example, but not limited to, growth containers while reference groups have been raised in growth containers not contacted with the test agents. In other embodiments, the flies are not wild type, but carry a genetic modification and are similarly contacted with the test agents for some groups and not for other, reference groups. In other embodiments, flies and other animals are to be screened for their behavioral response to test agents by any method known to one of ordinary skill in the art, for example by those methods described in U.S. Pat. No. 7,848,888, hereby incorporated in its entirety by reference.

In some embodiments of the invention, the device is used to test behavior in a test population of flies and a reference population of flies. In some embodiments, the test flies are wild-type flies. In some exemplary embodiments, the test flies carry one or more genetic modifications, in some embodiments the genetic modifications are transgenes, in some embodiments the modifications are genetics lesions made via non-transgenic methods, for example, but not limited, to ethyl methanesulfonate mutagenesis. In further embodiments, the test flies are transgenic for a gene encoding an RNA or protein with an effect on the normal behavior of the flies. In some embodiments, the gene affected by the modification(s) is homologous to a mammalian gene with an effect on psychiatric health in humans. In some embodiments of the methods of the invention, the test flies contain a genetic mutation resulting in a loss of function or a gain of function.

In some embodiments of the invention, a reference population of animal is used to control the effects of the test agents being used, either to contrast the effects of active test agents with what is expected in the absence of activity (negative control), or to compare the effects of test agents with the effects of agents known to have an effect (positive control) on the behavior under examination. These reference animals are in some embodiments, for example, (i) flies not treated with any test agent; (ii) flies treated with a compound or other agent that has a known effect on animals, for example, but not limited to, animals behavior; (iii) flies carrying a genetic modification (for example, transgene or lesion) and not contacted with a test agent, (iv) flies with similar genotype (and genetic background) as the test flies, and not contacted with test agent; (v) flies carrying a genetic modification and contacted with an agent of known effect; (vi) flies not carrying the genetic modification, but possessing an otherwise similar genetic background to the test flies and not contacted with a test agent; (vii) flies not carrying the genetic modification carried by the test flies but contacted with an agent on known effect.

Animals for use in the preferred embodiment invention are insects (members of the taxonomic group Insecta), for example, but not limited to, dipteran flies, though other animals (members of the taxonomic group Metazoa) may be used in other embodiments. Of particular use are flies of the taxonomic group Drosophila melanogaster during the imago (adult) stage, but other species and life stages are also appropriate for use in some embodiments. Also of particular use are flies possessing genetic modifications brought about by any method known to one of ordinary skill in the art, such as by use of genetic modifiers, including, but not limited to, P-element transposons, other transposons, engineered transposons, X-rays, ethyl methanesulfonate and other mutagens.

In each particular embodiment, the animals used in the present invention exhibit one or more traits that is indicative of and/or characterizes a change in behavior and/or brain function in the animal, in a manner that is similar to behavioral and psychiatric disorders in human or other target animals, such as, for example mice, rats, monkeys, dogs, cats, horses, pigs, cattle of any kind, pets, or farm animals. Hereinforth, all target animals are referred to as “humans.” The above changes in behavior include, for example, but are not limited to, impaired or improved motor skills, impaired or improved learning, altered locomotor patterns, etc. In some cases, test animals are flies which exhibit behaviors which have an evolutionarily conserved relationship with psychiatric or neurological disorders in human.

Genetic modifications possessed by the animals used the present invention may be incorporated into the test animals by any method known to one of ordinary skill in the art, for example, but not limited to, P-element transposons and their derivatives, PiggyBac transposons and their derivatives, other transposons, engineered transposons, homologous recombination, site-specific recombinases, X-rays, ethyl methanesulfonate (EMS), N-ethyl-N-nitrosourea (ENU), other mutagens and other methods of genetic modification some of which are described in (PubMed IDs) PMID 6289435, PMID 18641946, PMID 21831835, all hereby incorporated in their entirety by reference. The genetic modifications carried by the animals produce a behavioral phenotype different from that of wild-type animals.

In the subset of genetic modifications that involve transgenes (genetic material that has been inserted into the genome of a cell), it is in some cases of particular use to express the products of the transgene in a specific anatomical and/or temporal pattern within the animal to produce a phenotype relevant or specific to behavior, for example, but not limited to neuronal expression. Genetic material to be used in transgenes include RNA interference derivatives of endogenous material from the animal under study, material from exogenous sources, i.e. another species, for example a mammalian species, for example human genetic material. In some embodiments, the genetic material corresponds to, for example, a gene or genes implicated in directly or indirectly with a human psychiatric or neurological disease, in other embodiments, the material corresponds to a gene homologous to a human gene. In the embodiments where the transgenes are used as test agents, the genetic material corresponds to a gene of known or unknown relationship to a psychiatric or neurological disease.

In some embodiments, the transgene is integrated into the genome of the test animals and its expression is governed by a promoter or enhancers that define the anatomical range or temporal sequence of the expression of its products, such that the products are present in specific desired cell types and at specific desired developmental stages. The promoter or other expression control element may be endogenous, i.e. already present in the genome, exogenous, i.e. from the same species but introduced to a new location in the genome, heterologous, i.e. from another species, or synthetic. The expression control element may be physically close in the chromosome to the genetic material that generates the gene products (cis), or it may act at a distance, in some cases from a different chromosome, for example by means of a trans-activating element by any method known to one of ordinary skill in the art, for example by the GAL4-UAS system, the LexA-LexAop system or the Q system or another trans expression system PMID 8223268, PMID 16582903, PMID 12324939, PMID 20434990 all hereby incorporated in their entirety by reference.

In some embodiments, animals for use in the invention carry a non-transgenic genetic modification, i.e. mutation that lesions one or more of the endogenous genes to produce amorph, hypomorph, hypermorph, antimorph or neomorph alleles and thereby generates a phenotype. In some embodiments, the gene disrupted is homologous to a human gene, for example a conserved animal gene involved in psychiatric or neurological dysfunction, see, for example, PMID 11381037, PMID 9115203, PMID 18327252 all hereby incorporated in their entirety by reference. For use in the invention, non-transgenic modifications can be prepared by method known to one of ordinary skill in the art, for example, P-element transposition, x-ray irradiation, ethyl methanesulfonate or ethylnitrosourea PMID 6289435.

In some embodiments, the animals for use in the invention are wild-type flies that are treated with, for example, environmental stimuli, substances, nutritional deprivations and social manipulations that induce a disease-like state. As a non-limiting example, Drosophila rest responds to mechanically induced rest deprivation and show rebound rest in the interval following sleep deprivation PMID 10707978, hereby incorporated in its entirety by reference. Compounds that affect human sleep, for example caffeine and adenosine receptor ligands also show effects on fly rest and rebound rest, indicating conserved mechanisms PMID 10707978, PMID 10710313, both hereby incorporated in their entirety by reference. In some exemplary embodiments, the invention employs mechanical rest deprivation combined with test agent screening and behavioral analysis. As another non-limiting example, the flies are exposed to competitive conditions such as a potential mate or a food source, which amplifies fly aggression, PMID 11960020 hereby incorporated in its entirety by reference. This emotional behavior is influenced by genes that are conserved with human counterpart genes that also affect emotional behavior in human PMID 17450142. As another non-limiting example, the flies are treated to an environmental stress, for example, but not limited to hyper gravity, oxidative stress, dehydration etc. that affect behavior PMID 20161767, PMID 11707930.

In another exemplary embodiment, animals are treated with a substance for use in the invention, for example, wild-type animals contacted with a psychoactive and/or addictive substance. As an illustrative example, the flies are exposed to ethanol, inducing intoxication-like behavior or are presented with ethanol, inducing reward-like responses, PMID 9635429, PMID 21499254 both hereby incorporated in their entirety by reference. Other substances with conserved psychoactive effects between fly and human include cocaine PMID 10704411. Other non-limiting examples of chemically-induced models of human psychiatric or neurological disease in animals include, for example, those that target dopamine neurons with compounds such as 1-methyl-4-phenyl-1,2,3,6-tetrahydropyridine (MPTP) or 6-hydroxydopamine (6-OHDA) PMID 11331916 hereby incorporated in its entirety by reference. Yet further examples of substance-induced disease models in flies include cholinergic agonists, hydroxyurea and 5-hydroxytryptophan PMID 1679841, PMID 8303280, PMID 17450142, all hereby incorporated in their entirety by reference.

The above examples of animals to be used in the device are for exemplary purposes only, and may include another type of animal.

4.2. Bringing Test Agents into Contact with Animals

In the preferred embodiment, the flies are contacted with test agents in their growth containers, for example, but not limited to, by mixing test compounds with food. Flies are also to be contacted with test agents by any method known to one of ordinary skill in the art, for example by those methods described in PMID 17494737.

In other embodiments, flies are contacted with the test agents after removal from the growth chambers, for example, but not limited to, in containers after singulation. In other embodiments flies are brought into contact after removal from the growth containers and before singulation.

The term “test agent” is used to describe the agents being tested with a behavioral assay, though the effect of the agent on the behavior may be unknown. Test agents can be molecules, can be molecules produced by synthetic chemistry sources, can be isolated from natural sources, for example, but not limited to, viruses, marine and terrestrial microorganisms, algae, plants, fungi, prokaryotes, archaea, etc. Test agents, for example, but not limited to, are chemical compounds that are brought into contact with a group of animals such that the agent can be expected to affect a biological activity or a disease or disease process, in at least 75%, often at least 80% and in some embodiments as many as 95% or greater, if not all of, the animals.

Examples of test agents include, for example, drugs, pharmaceuticals, therapeutics, environmental, agricultural, or industrial agents, natural products, natural extracts, synthetic products, combustion products, pollutants, peptides, polypeptides, proteins, nucleic acids, proteins, sugars, salts, carbohydrates, gaseous molecules, chimeric molecules, benzodiazepines, oligomeric N-substituted glycines and oligocarbamates, organic compounds, inorganic compounds, purines, pyrimidines, derivatives, organic compounds, lipids, fatty acids, amino acids, steroids, glucocorticoids, antibiotics, flavonoids, structural analogs, stapled peptides, polymers, beta-turn mimetics, polysaccharides, phospholipids, hormones, prostaglandins, steroids, aromatic compounds, heterocyclic compounds, and/or combinations thereof. Test agents may also include non-chemical agents, for example, but not limited to, heat, cold, genetic manipulations, neural manipulations, optogenetic interventions, x-ray radiation, blue light, red light, light and electromagnetic radiation of other frequencies, radio waves, mechanical intervention, sound intervention, air flow, oxygen availability, the availability of other gases in the growth environment, water content of the growth media, density of animals in the growth container, the size of the growth container, the periodicity of the light/dark cycle, the compositional ratios of the growth media, the presence of conspecific animals, the presence of predatory animals, the presence of prey animals and other test agents with biological activity. Test agents are often assembled into large collections, termed libraries. Libraries of test agents and their preparation are well known to those of ordinary skill in the art, consisting both of natural products or synthetic products, and can be obtained from both public and private organizations, for example the National Institutes of Health, and private companies such as MicroSource Discovery Systems Inc. of Gaylordsville, Conn. which sells several collections including the Spectrum Collection which comprises more than 2000 compounds, including the compounds that form the National Institute for Neurological Disorders and Stroke (NINDS) NINDS Custom Collection II.

The present invention may also be applied to test agents with established pharmacological activity and derivative analogs thereof. Pharmacological agents with known biological effects may be modified to form close structural variants with chemical derivatizations, for example, esterification, amidification, oligomerization and acylation. Novel test agents are also generated using methods such as structure-guided drug design, computer modeling and molecular evolution to produce variants of known agents or novel agents with limited structural relationship to known agents.

For chemical compound test agents, the animals are brought into contact with the test agents such that the compounds are internalized. Internalization is, for example, but not limited to, through the digestive tract, i.e. via ingestion, in an aerosol or vapor via the respiratory system, for example, but not limited, to the tracheal system of flies, via passive transit through the cuticle or skin of the animals in the case of compounds able to traverse the external surface or through the external surface via injection or ballistic insertion. Animals may be contacted in any of the chambers of the device described herein, such as, for example, but not limited to, the growth container, any part of the singulation machinery, or the behavioral arena. The test agents may be introduced to the animal at any time during development. In the exemplary embodiments that use flies, these stages include but are not limited to the embryonic stage, the larval stage, pupation, metamorphosis, and the imago (adult) stage. Contact schedules between the animals and the test agents are facilitated by any method known to one of ordinary skill in the art, including, for example, acute or chronic contact. Assay mixtures may be run in parallel with different test agent concentrations to obtain a variety of responses suitable for comparison, for example, but not limited to, a negative control that includes no test agent may be incorporated into an assay or screen to better interpret the data.

### 4.3. Connecting the Growth Containers to the Intake Device

As is well known to one of ordinary skill in the art, existing fly cultivation containers are fabricated as, for example but not limited to, glass molded or plastic molded bottles, vials or multi-well plates and filled with a source of food for flies. The preferred embodiment of the presently described invention takes in flies from a reservoir of multiple such fly containers, such that multiple containers are loaded into the device to accommodate prolonged or continuous operation. The container used to store the flies is sealed to prevent fly escape with a closure that is compatible with the intake port of the singulator. In this preferred embodiment, the containers are sealed with a closure containing a self-closing spring lid similar to that described in U.S. Pat. No. 4,679,700 and in common use in trash receptacles. FIG. 1 shows a preferred embodiment for connecting fly growth containers to the intake device. In this embodiment, the growth containers are represented as fly growth bottles (1) that have been placed on a conveyor belt (2) that moves to place a bottle under the fly intake tube (3). The intake device is mounted on a linear actuator that lowers it into the growth container, which is sealed with a spring-loaded closure that can be pressed open via the descending intake tube. In this way, the flies are able to climb into the intake tube due to their negative geotactic behavior as described in PMID 5229969 and PMID 5787804, both hereby incorporated in their entirety by reference. A closure may be constructed according to any designs known to one of ordinary skill in the art to the effect that the intake tube can access the container, where the flies remain contained. As a non-limiting example, the growth container closure contains a hinged flap similar to that in U.S. Pat. No. 4,679,700, hereby incorporated in its entirety by reference, that is opened by the intake tube (3) to gain access to the flies, and is pushed closed by a spring as the intake tube retracts.

In one embodiment, the containers are moved into position by a conveyor. In another embodiment, the containers are moved into place by a robotic arm. In another embodiment, the flies are cultured in a large chamber and are transferred into an antechamber from which the intake tube receives flies. In an embodiment of the latter embodiments above, the flies are transferred into the antechamber by suction drawing from the antechamber through a perforated surface that blocks the flies from leaving the antechamber but that allows air flow. In another such embodiment the mass growth chamber is connected to the antechamber via a gate. In another such embodiment the gate is moved by, for example but not limited to, a solenoid.

In addition to the exemplary embodiment of a spring-loaded lid on the growth container, in other exemplary embodiments, the closure is a thin film that is punctured by the intake port. In other embodiments, the closure is a lid that is pushed aside as the container is moved into place adjacent the intake tube.

In another embodiment, the flies are anesthetized in the growth container, and then transferred while inactive to the intake tube and/or the singulation device. Animals are anesthetized by any method known to one of ordinary skill in the art. In a particular embodiment, the device to mediate this is itself an robotic system such as, for example but not limited to, the one described in U.S. Pat. No. 6,688,255 hereby incorporated in its entirety by reference. In another embodiment the method of anesthesia is carbon dioxide applied to growth containers, such as in the device described in U.S. Pat. No. 4,106,438 hereby incorporated in its entirety by reference. In another embodiment the flies are anesthetized via a injection of diethyl ether into the growth container. In another embodiment, the flies are anesthetized in the growth container via chilling the container to a temperature sufficient to immobilize the flies to temperatures between −15 C and 30 C degrees, more preferably between −10 C and 15 C, more preferably between −5 C and 5 C, and most preferred to temperatures between −2 C and 2 C. In another non-limiting example, the flies are anesthetized in the growth container via injection of another gas containing low levels of oxygen, for example but not limited to nitrogen, argon, xenon or any other appropriate gas or mixture thereof.

In other embodiments, the flies are transferred from the growth container to the singulation device in a similar manner to the fly stock transfer robots manufactured by Keller Smartwood Engineering of Aurora, Oreg. In this exemplary embodiment, the growth container is turned on its side and injected with anesthetic gas, and the immobilized flies are pushed out of the growth container with a second gas puff into a second container. In embodiments using this method, the second container is the singulation area.

In other embodiments, the flies are sorted as embryos into separate growth containers, via, for example, but not limited to, the device described in PMID 11175730 hereby incorporated in its entirety by reference. In this embodiment, the flies are either singulated or sorted into groups with defined numbers of individuals, and are on growth media required to develop from embryo to adult fly. In one embodiment the pre-sorted flies are assayed for their behavior in their growth container. In one embodiment, the embryo-sorted flies are moved into the behavioral arena by lowering an intake tube into the individual chamber and allowing the fly or flies to crawl into it. In another embodiment, the pre-sorted (individual or grouped) flies are anesthetized before transfer into the behavioral arena.

In other embodiments, the intake tube is connected to a source of suction and active flies are vacuumed into a container in which the passage of flies into the suction source is blocked by a fine mesh, for example, but not limited to, fine woven nylon mesh. In such embodiments, flies are held in place by the suction until transferred to the singulator. In one exemplary embodiment, the flies are anesthetized while in the mesh chamber.

In other embodiments, the intake device itself plays a role in singulation, for example but not limited to, a device it inserted into the growth chamber, that contains one or more small passageways that only accommodate animals in single file, allowing their passage out of the growth container in an arrangement that facilitates further singulation or separation into smaller groups. The animals walking in single file through the narrow intake is then used in one of several possible separation/singulation mechanisms, for example but not limited to, some of the singulators/separators described herein, for example but not limited to, a y-junction separator, a conveyor belt system referred, a vacuum pipet singulator or separator, a gated grid separator, a gated air flow separator.

In other embodiments of the methods of the invention, animals are transferred from the growth container via means of a pipet. For example but not limited to, animals are transferred via a vacuum pipet directly from a growth container either to an anesthesia device or directly to a behavioral arena. In one embodiment, the pipet transitions from vacuum suction to intake the flies from the growth container to positive pressure to expel the flies from the pipet into the target location.

### 4.4. Preparing the Animals for Singulation

In the preferred embodiment, prior to singulation (separation into single individuals), fly preparations may be prepared. As a non-limiting example, the number of flies may be set, controlled, or adjusted. As another non-limiting example, flies may be anesthetized at this step. In a preferred embodiment, the flies are removed from the growth container in large numbers while active. The intake tube (3) is connected to an intake cassette (4), which is placed adjacent to an anesthesia chamber (8). The flies are allowed to crawl out the growth container, up the intake tube (3) and into the intake cassette (4). The intake cassette is open on the side facing the anesthesia platform (8), so the flies are free to walk onto the anesthesia platform (8). The ceiling of the anesthesia chamber (not shown in the FIGURE for clarity) is fabricated from clear material, such as, but not limited to, glass or plastic to allow inspection of the chamber from above. In the preferred embodiment, the floor of the anesthesia chamber is fabricated from a peltier thermoelectric device. The floor of anesthesia chamber (8) is fabricated from a peltier device which is off while the intake port is inserted into the growth container. When appropriate, the peltier device is turned on to lower the temperature of the platform to, for example but not limited to, zero degrees centigrade, causing the flies to become inactive. In an exemplary preferred embodiment, the number of flies and their activity are monitored via the video camera (27) attached to one of the rails of the robotic arm (25). In another exemplary embodiment, the camera is fixed at a position from which the motion of the robotic arm is monitored. The camera is connected to a computer running machine vision software to count flies and measure their walking speed. When the flies have stopped moving, the anesthesia platform and the wall opposite the intake cassette (30), which is attached to the platform are translated laterally on the anesthesia platform conveyor (9) to the fly picking position. The platform is translated on a horizontal mechanical conveyor. As two other walls and the ceiling are attached to the device bed (26), or are part of the intake cassette (4), this action results in exposing the flies to access from above and puts them in reach of the fly pipets (20).

In another embodiment the intake cassette is not simply open, but constructed to contain a constricted outlet facing the anesthesia chamber, thus limiting return of the flies to the intake cassette from the anesthesia chamber. In one embodiment, the constricted outlets are preceded by narrowing of the interior of the intake cassette. In this manner, flies are drawn into an increasingly constricted passageway until they must form a single file before exiting the constricted outlet to the anesthesia chamber. The small size of the intake cassette outlet reduces the likelihood or possibility of the flies to re-enter the intake cassette.

In another embodiment, the intake cassette has a source of positive air pressure on the side distal to the anesthesia chamber. This has the effect of pushing the flies towards the anesthesia chamber. In an embodiment that combines the constricted outlet from the intake cassette and air flow from the distal side into the anesthesia chamber, further increasing the likelihood that the flies will enter, and remain in the anesthesia chamber. In another embodiment, the anesthesia chamber is illuminated by a bright light, making use of flies' positive phototaxis behavior and inducing them to approach. In yet another embodiment, the anesthesia chamber is connected to a source of negative air pressure that is obstructed to prevent intake of flies. In one embodiment of this device, the walls are the source of negative air pressure and are constructed with perforations smaller that the size of a fly so that the flies are drawn into the anesthesia chamber by the air flow, in a similar manner to that described in U.S. Pat. No. 3,965,608 hereby incorporated in its entirety by reference, but not into the source of negative air pressure. In yet another embodiment, perforated walls are a source of gentle positive air flow in which the air has been brought into contact with an attractive odorant; in this embodiment the attractive odor draws the flies into the anesthesia chamber.

In another embodiment, the floor of the anesthesia chamber is constructed of hollow material through which coolant can be delivered; the flies' temperature is lowered by transporting the coolant to the interior of the hollow platform. This is achieved by any method known to one of ordinary skill in the art, including, but not limited to, an aluminium platform through which cold water is pumped. In another embodiment, the anesthesia chamber contains perforations that are used to exude an anesthetic gas, such as but not limited to carbon dioxide, diethyl ether vapor, nitrogen, argon, xenon or another appropriate gas or mixture thereof, in a similar manner to that described in U.S. Pat. No. 4,106,438 hereby incorporated in its entirety by reference. In another embodiment, the anesthesia chamber is made accessible to a needle conveying a source of anesthetic substance, for example but not limited to, diethyl ether in a needle as described in U.S. Pat. No. 4,224,898 hereby incorporated in its entirety by reference.

In other embodiments, removing the flies from the growth chamber and placing them at the next location, for example but not limited to, onto an anesthesia pad or into a behavior arena is done by a pipet. The pipet is inserted through the cap of the growth container and, for example but not limited to, vacuum suction is applied to draw in flies. In some embodiments the pipet is then subjected to anesthesia treatment, for example but not limited to, rapid cooling by cold air or by contact with a chilled environment or, in other exemplary embodiments, by introduction of anesthetic gas into the pipet. The anesthetized flies are held in place with negative suction in the pipet and then deposited to the next location, for example but not limited to, the behavior arena. In other embodiments, the flies are held inside the pipet by negative pressure and are transferred directly to another location, for example but not limited to, a behavior arena or an anesthesia chamber. In some such embodiments, retention of the flies in the pipet is aided by the conical shape of the pipet, such that the opening of the pipet experiences a high flow rate through relative to the flow rate inside the remainder of the pipet cavity.

### 4.5. Clearing the Animal Intake System and Anesthesia Chamber

In the preferred embodiment, if not cleared, the intake device will only be able to process a single container of flies before manual clearing and cleaning becomes necessary. As prolonged or continuous operation is desirable, in the preferred embodiment the intake system is cleaned with, for example, but not limited to, vacuum to clear the flies left behind in the intake system and singulator (i.e. not picked up by the fly pipet for relocation to the behavior arena). In the preferred embodiment, the intake tube (3) and intake cassette (4) are moved to a suction intake port 7 that draws the flies remaining in the intake system to waste. In this situation, the flow of flies is from the intake cassette (4) to the intake tube (3) and then into the suction tube (7). The intake system is connected to a robotic arm (8) mounted on rails (10). The robotic arm is moved up out of and down into the growth container and laterally between the current growth container position 1 and the suction tube to waste which is at the same level as the accessible interior of the growth container. To clear the anesthesia chamber, there is a second rectangular cassette, the cleaning cassette (5), that is mounted on another branch of the branched robotic arm (28). As the robotic arm moves the intake tube to suction, the cleaning cassette (5) is concomitantly move into place adjacent to the anesthesia chamber. The suction connector (29) for this cassette will, as a result, be placed on top of a high-power suction source (6). This arrangement will remove the inactive flies in the anesthesia chamber by suction, as the flies are drawn from the anesthesia chamber (8) into the cleaning cassette (5) and suction connector tube (29) and then into clearing suction tube (6).

In other embodiments the intake system is cleared by other methods, for example but not limited to, flash-cooling by, for example but not limited to, flushing with a gas, flushing with air, flushing with cold air, or via mechanical agitation to dislodge the animals from the walls of the intake system.

### 4.6. Singulating the Animals

In the preferred embodiment the anesthesia platform is placed under the fly pipetting robot, which includes an array of independently movable fly pipets (20), a vertical robotic arm (11), a horizontal robotic beam (25), a carriage connecting vertical arm and horizontal beam, a device that conveys the arm along, a rear horizontal rail (17) and a front horizontal rail (24). This arrangement of robot arms is similar to that described in U.S. Pat. No. 6,264,419 hereby incorporated in its entirety by reference and allows three-dimensional positioning of a pipette or other tool. To acquire a map of the anesthetized flies' positions, the robot arm is positioned to place the camera directly above the anesthesia platform where it collects an image. Machine vision software is then used to identify single flies and their positions in a similar manner to identifying colonies on Petrie dishes during the operation of colony picking robots such as those described in Uber et al. (Biotechniques, vol. 11, No. 5, 1991, pp. 642-646), Jones et al. (Nucleic Acids Research, vol. 20, No. 17, 1992, pp. 4599-4606), WO 1992/012233 A1, EP 1421994 B1, U.S. Pat. No. 7,776,584, EP 1421994 B1, U.S. Pat. No. 5,587,062 and U.S. Pat. No. 6,658,324 all hereby incorporated in their entirety by reference. Instances of this kind of robot include the QPix brand robot sold by Genetix of New Milton, United Kingdom and the Pickolo brand colony picking robot sold by SciRobotics of Kfar Saba, Israel that works in conjunction with robots made by Tecan of Mannedorf, Switzerland. An image is taken of the anesthesia pad, and this image is used to identify the flies' locations. Once a map of the flies' locations has been made, this is then used to guide the actions of the fly pipet array, the members of which are independently movable in a similar manner to the picking needles described in U.S. Pat. No. 6,658,324 hereby incorporated in its entirety by reference. When a single fly is identified in the map, the robotic arm positions a fly pipet directly above it. The fly pipet is then lowered towards the anesthesia platform (8) when it is located at the extended end of the anesthesia platform conveyor (9). The tip of each fly pipet is constructed as a cylinder. The section just above the end of the cylinder is occluded with a layer of nylon mesh, for example but not limited to, Nitex brand mesh. The pipet is connected to source of negative pressure that is switchable via, for example but not limited to, a solenoid valve. Aspects of this device are similar to the devices described in U.S. Pat. No. 4,822,022 and U.S. Pat. No. 3,965,608, both hereby incorporated in their entirety by reference, and also bears similarity to the mouth pipet used by Drosophila researchers to sort flies for genetic crosses, a device and technique widely known to those skilled in the art. As the pipet approaches the fly lying on the anesthesia pad, the fly is drawn up into the cavity inside the cylinder by the air flow and held against the mesh. This is repeated with each of the pipets at the end of the arm (20). In this manner, a group of anesthetized flies can be isolated into single animals for delivery to behavioral arenas.

While the above shows a preferred embodiment for a fly singulator, there are other embodiments of a fly singulator. In another embodiment, a device similar to the intake cassette takes on a role of singulator, functioning to allow the flies to walk in single file along a narrow tube; flies are isolated into a single-file tube as preparation for singulation. In this embodiment, the path from the cavity inside the intake cassette starts to narrow as the flies travel away from the intake tube until the path is a small tube that can only accommodate a single fly. The flies are induced to enter narrow tubing, so as to produce a single column of flies by any method known to one of ordinary skill in the art, including, but not limited to the flies' own locomotor behaviour. In one embodiment, the material surrounding the single-file path is fabricated from clear material. This is inspected by a video camera linked to a computer running machine vision software to identify the location of flies in real time, such as for example but not limited to the software described in PMID 19837039, hereby incorporated in its entirety by reference. As a non-limiting example, to singulate two flies, the path is punctuated with two controllable gates, implemented by any method known to one of ordinary skill in the art, including, but not limited to, for example but not limited to, thin plastic discs linked to a solenoid controlled by virtual instrument software on a computer. As the flies proceed along the path, the video is analyzed to find when the leading fly passes the first gate, but is not immediately preceded by another fly and the gate is closed behind it. The second, downstream gate is left unclosed until the first fly passes it, at which point it is closed. At this point, the first gate is reopened, allowing a second fly to walk into the space between the two gates, whereupon the first gate is closed, leaving the two flies singulated, separated from each other and the other flies by gates. In another embodiment, there is a longer series of gates, for example but not limited to between 3 and 10 gates, between 11 and 100 gates, between 101 and 1000 gates and/or more gates. In one embodiment, for example but not limited to, to transfer the flies into the behavioral arena, there is another gate downstream of the first fly, that is opened to release the fly into the space beyond, where the behavioral arena has been located. Once the fly walks into the arena, the arena is closed and the terminus of the gated singulator is reposition to the next arena (or vice versa: the arena plate is repositioned to place an empty arena adjacent to the outlet of the singulator). In another embodiment of the path is branched into, for example but not limited to, two or three dimensions, thus increasing the number of fly gating operations by the number of paths; as flies walk from the intake tube, they approach forks in the path and begin spreading themselves out along the various gated singulator paths. In one exemplary embodiment, the branched paths terminate in an arrangement that is made to have the same spacing as the arenas in the behavioral plate. In this way, when the terminal gate for each branch is opened, each arena should receive a fly. In another embodiment, the flies are induced to walk along the path towards the terminal gate using any method known to one of ordinary skill in the art, including, but not limited to relying on the innate exploratory behavior of flies, placing a light near the path terminus to induce positive phototactic behavior, pumping an attractive odor along the length of the path through perforations in the gating discs to induce attracted chemotaxis, angling the path upwards to induce negative geotactic behavior, drawing an aversive odor from the side of the intake tube to induce avoidance chemotaxis, drawing the flies into the narrow entrance with suction.

The tubing in the singulator is manufactured by any method known to one of ordinary skill in the art, including, but not limited to fabricated cylindrical tubing, tubing patterns cut from solid sheet material and laminated to form paths, tubing machined or otherwise cut from solid materials, tubing fabricated from molded materials, 3D printed material containing tubes or tubing from rolled materials. In a preferred embodiment the tubing is machined, printed or molded from transparent plastic that is resistant to exposure to flies. In one embodiment, one surface of the path is constructed as a single sheet of clear material, for example but not limited to glass or transparent sheet plastic. This embodiment allows inspection of the flies, removal of the plate for access and cleaning and access for transfer to the behavioral arena.

In another embodiment, the tube is connected to the narrow end of a funnel. In one preferred embodiment, the funnel is used by allowing animals, for example, but not limited to, flies to fall by gravity into a restricted diameter tube. In another embodiment, the single-file tube is sufficiently narrow to prevent the animals from turning around or backing up. In yet another embodiment, the diameter is used to exclude one sex but not the other, for example, but not limited to, female flies are prevented from entering the tube, while the smaller males are able to enter.

In other embodiments, the flies are separated for singulation in a series of iterative inverted y-junctions wherein by gravity the fly will fall either of two directions as it encounters a Y-junction, for example but not limited to, branched tubing formed within, for example but not limited to, an acrylic manifold such as those manufactured by Connecticut Plastics of Wallingford, Conn. In some embodiments, the animals are shaken from the growth container into the apical opening of the first y-junction, while the manifold is mechanically agitated, thus preventing the animals from grabbing hold of the walls and introducing desirable stochastic property into the descent of the animals through the manifold. In some embodiments, the branching y-junctions terminate in an array that matches the array of target locations, for example but not limited to, the behavior arena and in this manner are delivered to the behavior arena. In some embodiments, the animals are delivered in an imprecise manner, with each target location receiving a random number of animals around the mean. In this manner, experiments are to be performed on the various numbers of animals that are delivered to each behavioral arena. In other embodiments, the termini of the y-junction branches are gated with, for example but not limited to, a solenoid gate or a hydraulic gate and the target locations are monitored with a machine vision system that scores when the location contains one or more animals. The gates of the termini are closed when the corresponding target location contains, for example but not limited to, one or more animals, thus ensuring a more consistently even distribution of animals in the target location. In these embodiments, the variability of animal density in each arena will benefit the analysis by providing results that cover a range of interactions, ranging from a solitary animal to larger groups.

In other embodiments, the flies are isolated from each other by a series of y-junctions in the tube as described above, and the motive force is the animal's own mobility rather than gravity. As animals walk into the branching tubes, whereupon they become increasingly isolated from the other flies as the number of branches increases and the flies approach the far end of the branches. Once at the end of the branched structure, the individual flies can be transferred to the behavioral arena. In some embodiments, the branches increase as the animal ascends, and the animals are drawn upwards by, for example but not limited to, negative geotaxis, positive phototaxis brought about by a light from above.

In some embodiments, the flies are diluted into single containers. In these embodiments, for example but not limited to, a pitted platform is used—the pits are shaped to allow only one fly in each. Flies are delivered to the platform, anesthetized and the platform is shaken until all/most wells contain a fly, then platform is cleared with a soft brush. In some embodiments employing active flies, the single-fly pits are narrow and contain a small amount of food at the bottom. The food pits are spaced in the same periodicity as the behavioral arenas. Flies are added en masse and allowed to crawl into the pits. After a given time optimized to recruit flies into the pits, the excess flies are removed by either an air puff, suction or a combination thereof, and the capture pit array is brought into contact with the behavioral arena.

In other embodiments, singulation is done via a funnel or V-slide in a manner similar to a shrimp singulator described in U.S. Pat. No. 4,692,965, hereby incorporated in its entirety by reference. In these embodiments, flies are anesthetized and placed en masse into a funnel or V-slide, the outlet of which is small enough to only permit one fly at a time, and which is attached to vibratory driver motor. As the singulator is vibrated, the single flies are delivered to a target below, which in some embodiments is the behavioral arena. The behavioral arena plate is moved, once a fly has been determined to be delivered to the most recent arena.

In yet other exemplary embodiments, the singulator is constructed using three conveyor belts placed at cross-angles to each other and running at progressively higher speeds, a device is similar to one manufactured by Applied Robotics of Glenville, N.Y. In this embodiment, the flies are anesthetized using one of the singulation preparation methods described above, and then dispensed in bulk onto a slow-moving conveyor belt that terminates at a ramp that is closely fitted to the curve of the first conveyor and delivers the flies to a second conveyor belt running at a right angle to the first. As the flies slide from the first conveyor to the second, faster conveyor, they bump into a wall built on the far side of the belt where they are both spread out and aligned along the wall. The second belt has a smooth plate mounted at a slight angle, for example but not limited to, between 2 degrees and 44 degrees to the trajectory of the anesthetized flies. Hitting this plate subjects the flies to alignment along this plate and prepares them for another plate mounted a slight, but opposite angle to the flies' trajectory. Flies aligned into a single column this way are then be dispensed into the behavioral arenas using any method known to one of ordinary skill in the art, including, but not limited to, for example but not limited to the fly pipetting robot describe above.

### 4.7. Placing the Animals in the Behavioral Arenas

In the preferred embodiment, once the fly has been singulated into the pipet the robotic pipet arm actuators (11, 14, 25) transport it to the behavioral arena plate (13), where it drops the fly into the arena by switching off the suction air flow, allowing the fly to fall. The behavioral plate (13) is sitting on a chilling platform (12) that, like the preferred embodiment of the anesthesia platform, chills the plate to a temperature sufficiently cold to keep the flies immobile, for example but not limited to zero centigrade. This procedure is repeated until the pipetting robot has delivered a fly to each arena within the behavioral multiplex plate. The pipetting camera (27) mounted on the pipetting robot is then positioned to be above the behavioral plate (13) to verify that each well has received a fly. If a fly has failed to be delivered to an arena, the pipetting robot will then return to retrieve another fly. In drawing of the preferred embodiment, the pipetting robot is shown to have an array of four pipets (20), allowing the robot to collect four flies from the anesthesia platform for each trip to the behavioral arena.

In other embodiments, the flies are delivered to the behavioral arena via system of tubes carrying gated, controlled air flow. Flies are isolated into single file either by active locomotion or by singulation using one of the described embodiments above. In some exemplary embodiments, two flies are to be delivered to two arenas. As the first fly approaches the first gate, a gate on one side of the fly is triggered to open, while simultaneously air is puffed from the opposite side. This action works to place the fly in a unique location, which is to be used as the behavioral arena. When the second fly approaches this same gate, the gate is not triggered as this location already has a fly, and the fly is allowed to progress to the second gate, whereupon it is drawn into the second arena. In yet other embodiments, there are more than two behavioral arenas. In another embodiment, the gate uses negative pressure to draw the fly into the arena.

### 4.8. Submitting the Behavioral Arena Plate to Inspection

In the preferred embodiment, once the flies have been delivered to the behavioral arena plate (13), the plate gripper (15) attached to the plate handling robot (16) approaches the stack of lids (22). The plate gripper is controlled along three axes, much like the fly pipetting robot, and the gripper can be rotated around the vertical axis to allow positioning the plates the different orientations, in a similar manner to that described in U.S. Pat. No. 6,264,419, hereby incorporated in its entirety by reference. Robot systems like this are sold by Tecan of Switzerland, and other robotic grippers with similar functionality are well known to those of skill in the art. The gripper (15) removes a lid from the stack, transports it to, and places it on the behavioral arena plate (13). After this, the gripper lowers to grab the bottom of the behavioral plate, and lifts both plate and lid away from the plate chiller. The plate handling robot then conveys the plate to one of the inspection positions (for example 18a) on the main platform (26). Inspection position (18a) is located directly beneath a camera, (19a). Also shown in the drawings is a second inspection position (18b) and camera (19b). As the flies recover from anesthesia, the camera records their locomotor behavior and sends the video frames to a computer, where they can be analyzed either online or offline for behavioral traits, for example, but not limited to, walking speed, circadian activity cycles, sleep/wake behavior, responses to odorants, aggressive motor actions, courtship motor actions, social interactions etc. Behavioral detection and analysis by videography and machine vision is well known in the art, including methods described in U.S. Pat. No. 7,848,888, PMID 19837039 and PMID 19412169 all hereby incorporated in their entirety by reference. In the preferred embodiment, two cameras (19a, 19b) are shown as an example.

In some exemplary embodiments, the behavioral arenas are fabricated from, for example but not limited to, polystyrene, polycarbonate, polypropylene or other plastic or solid material with outer dimensions that conform with standard microtiter plate formats, well known to those of skill in the art. In these embodiments the use of a standard plate format allows integration with existing, widely-used liquid handling robots commonly used for manipulation and dispensation of chemicals.

In another aspect of the invention, a record of the flies' location is maintained during inspection by any method known to one of ordinary skill in the art, including, but not limited to the following embodiments. In some preferred embodiments, the behavioral arena plate is transparent and is positioned below a video camera that is connected to a computer running machine vision software that tracks the location of flies. In a preferred embodiment, the plate is illuminated by light with little or no effect on the flies' behavior, for example, but not limited to, an infrared light source.

In other embodiments, the presence of a fly in a region of interest is detected by a localized fly detector, by any method known to one of ordinary skill in the art, including, for example, but not limited to an emitter-detector LED-photodiode pair placed on either side of the arena, or alternatively, the presence of a fly at a location in the tubing is detected by an ultrasound monitoring device PMID 10710313, hereby incorporated in its entirety by reference.

### 4.9. Behavioral Plate Removal and Continued Operation

In the preferred embodiment, the device is operated for extended periods of time, so that flies from a series of bottles are subjected to behavioral analysis. Once behavioral analysis of a plate is completed, the plate is removed from the inspection position by the gripper (15) and transported to the disposal opening (23), where it is dropped into a waste container. This operation allows another plate to be moved to the inspection position once the empty plate has been moved from the plate stack (21), loaded with flies, and then closed with a lid drawn from the lid stack (22).

In the preferred embodiment, the device is operated for extended periods of time, so that flies from a series of bottles are subjected to behavioral analysis. Once behavioral analysis of a plate is completed, the plate is removed from the inspection position by the gripper (15) and transported to the disposal opening (23), where it is dropped into a waste container. This operation allows another plate to be moved to the inspection position once the empty plate has been moved from the plate stack (21), loaded with flies, and then closed with a lid drawn from the lid stack (22).

In some embodiments, the waste container is chilled to prevent escape of active flies. In another embodiment, the waste container is filled with an anesthetic or lethal solution, for example ethanol, well-known in the art as a liquid used to dispose of flies. In yet another embodiment, instead of being dropped into a waste container, the gripper robot moves the plate to a device that removes the flies from the plate, for example, but not limited to, a source of negative pressure that draws the flies in while the lid is being removed. In this manner, the plate can be loaded with flies again and re-used.

### 4.10. Pharmaceutical Compositions

The invention provides a system for the identification of agents and pharmaceutical compositions that have activity to affect animal behaviors, including the behavior of flies and the behavior of mammals, for example, but not limited to, humans. The compounds or other test agents identified via the use of the present invention can serve as leads for further derivatization and development into a therapeutic, or the agents themselves can themselves be used as therapeutic agents, for example, but not limited to, therapeutic drugs in other animals. The invention further provides for the use of pharmaceutical compositions and other agents identified with the screening system for the treatment of disease in humans and other mammals, for example, but not limited to psychiatric and neurological illnesses. In some embodiments, the invention is a method of preparing compounds for treatment of disease in animals by contacting flies with candidate compounds, using the methods and devices described herein to prepare single flies or groups of flies in a manner so as to measure the resulting behavior of the flies, and formulating identified compounds for use with other animals, for example, but not limited to, humans.

The invention claimed and described herein is not to be limited in scope by the specific embodiments herein disclosed since these embodiments are intended as illustrations of several aspects of the invention. Indeed, various modifications of the invention in addition to those shown and described herein will become apparent to those skilled in the art from the foregoing description. Such modifications are also intended to fall within the scope of the appended claims.

Several references are cited herein, the entire disclosures of which are hereby incorporated, in their entirety, by reference herein.

### 5. Example 1

### High Throughput Screening of Compounds that Affect Circadian and Sleep/Wake Behavior

The presently described invention is used to screen compounds that influence the sleep and wake behavior of flies, by any method known to one of ordinary skill in the art, for example, but not limited to preparing flies for behavior analysis with the methods described in PMID 19369499 and PMID 20075256, both hereby incorporated in their entirety by reference. Potential drug targets influencing circadian and sleep/wake behavior are conserved between fly and human.

### 6. Example 2

### High Throughput Screening of Compounds that Affect Learning Behavior

The presently described invention is used to screen compounds that influence the olfactory learning behavior of flies, by any method known to one of ordinary skill in the art, for example, but not limited to preparing individual flies for behavior analysis with the methods described in PMID 119837039, hereby incorporated in its entirety by reference. Potential drug targets influencing learning behavior are conserved between fly and human.

1. A method of identifying compounds that affect animal or human behavior, said method comprising the automated steps of
1) preparing test animals;
2) separating the test animals
3) contacting animals with compounds
and further comprising a subsequent process of subjecting animals to behavioral analysis.

2. The method of claim 1, wherein the process of subjecting animals to behavioral analysis is automated.

3. A method of preparing animals for behavioral analysis by transferring animals from a growth container to a behavioral arena, said method being applied by use of a device that comprises a mechanism that separates test animals and a mechanism to dispense separated animals.

4. The method of claim 3 wherein the used device further comprises an anesthesia system.

5. The method of claim 3 or 4 wherein the device further comprises a behavioral arena positioned to be the subject of a behavioral recording device.

6. The method of claim 5 wherein the device is further connected to a device that delivers sensory stimuli to the test animals

7. The method of claim 5 or 6 wherein the behavioral recording device or the devices delivering sensory stimuli are under automated control.

8. The device of claim 3.

9. The device of claim 4.

10. The device of claim 5.

11. The device of claim 6.

12. The device of claim 7.

13. The compound identified in claim 1 or 2.

14. A pharmaceutical composition of a compound of claim 13.
